(19) United States
(12) Patent Application Publication
(10) Pub. No.: US 2010/0268077 A1
(43) Pub. Date: Oct. 21, 2010

(54) DIAGNOSTIC SYSTEM FOR CARCINOGENICITY AND DIAGNOSTIC METHOD FOR CARCINOGENCITY

(76) Inventors: Eizo NAKAMURA, Tohaku-gun (JP); Akio Makishima, Tohaku-gun (JP); Kazunori Okabe, Tohaku-gun (JP)

Correspondence Address: BIRCH STEWART KOLASCH & BIRCH, PO BOX 747, FALLS CHURCH, VA, 22040-0747, US

(21) Appl. No.: 12/761,053

(22) Filed: Apr. 15, 2010

Related U.S. Application Data
(60) Provisional application No. 61/169,965, filed on Apr. 16, 2009.
(60) Provisional application No. 61/176,293, filed on May 7, 2009.

(30) Foreign Application Priority Data
Jul. 27, 2009 (JP) P2009-171178

(51) Int. Cl. A61B 6/00; G01T 1/02; G01T 1/161

(52) U.S. Cl. 600/436; 250/363.03; 250/393

(57) ABSTRACT

A diagnostic system for carcinogenicity diagnoses a risk of developing cancer due to a radioactive material deposited in a human body. The system includes a detecting apparatus that detects radiations emitted from the radioactive material; and a determining apparatus that obtains a dose of the radiations detected by the detecting apparatus and determines the risk based on the dose.

### BACKGROUND OF THE INVENTION

The present invention relates to a diagnostic system for carcinogenicity and a diagnostic method for carcinogenicity that diagnose the risk of developing cancer due to a radioactive material deposited in the human body.

In recent general cancer screening, a testing apparatus using positrons, called a positron CT (or PET), has been used extensively.

In the positron CT, isotope-tagged glucose is injected into the human body and the distribution of the glucose within the human body is transformed by a special camera into an image. Using the fact that the amount of glucose taken up by cancer cells is larger than that by normal cells, cancer cells are identified. However, some types of cancer are difficult to identify even by use of the positron CT and thus the positron CT is not versatile.

Meanwhile, a cancer diagnostic support system such as that shown in Patent Document 1 is known. Specifically, when a tumor tissue sample is collected from a cancer patient and the sample is subjected to biopsy or cytology, if a determination just based on a threshold is made, an erroneous determination may be made for those samples near the threshold. Thus, in the support system, a reference range for making a sample determination is extracted from a plurality of sample data units and canceration of a sample can be determined while clinical information in sample data in the reference range is referred to.

Patent Document 1 is a Japanese Laid-Open Patent Publication No. 2009-072181.

### SUMMARY OF THE INVENTION

Conventional diagnostic systems can detect cells that have or are suspected as having become cancerous, but cannot detect potential cells that may become cancerous.

An object of the present invention is therefore to provide a diagnostic system for carcinogenicity and a diagnostic method for carcinogenicity that can diagnose the risk of cells becoming cancerous due to a radioactive material.

The inventor of the present invention has reached the following findings in the course of the study of pleural mesothelioma which is considered to be caused by asbestos.

Specifically, when iron-containing amphibole asbestos is inhaled into the lungs, any one of ferritin, which is known as an iron-binding protein, and hemosiderin, which is a partial degradation product of ferritin, or both is(are) deposited on the asbestos, forming asbestos bodies. On the asbestos bodies, a radioactive material such as radium is deposited. Moreover, the radium that is deposited on the asbestos bodies is accumulated in considerable amount in a relatively short period of time and thus radiation may be emitted at high frequency. In particular, since the asbestos bodies are in close contact with tissues such as lungs in the human body, radiation emitted from the radium is directly irradiated to cells, which may significantly promote canceration of the cells.

The inventor of the present invention has reached the following idea. Specifically, by detecting radiation which is emitted from radium deposited on an asbestos body, the fact that radium is deposited in the human body is detected, whereby the risk of a cell becoming cancerous due to the radiation emitted from the radium can be found. By this, the inventor has arrived at the present invention.

A first invention is directed to a diagnostic system for carcinogenicity that diagnoses a risk of developing cancer due to a radioactive material deposited in a human body, the system comprising: a detecting unit that detects radiations emitted from the radioactive material; and a determining unit that obtains a dose of the radiations detected by the detecting unit and determines the risk based on the dose.

A second invention is directed to a diagnostic method for carcinogenicity that diagnoses a risk of developing cancer due to a radioactive material deposited in a human body, the method comprising: a detection step of detecting radiations emitted from the radioactive material; and a determination step of obtaining a dose of the radiations detected in the detection step and determining the risk based on the dose.

According to the present invention, radiations emitted from a radioactive material deposited in the human body are detected and a dose of the radiations is obtained. Then, based on the dose, the risk of developing cancer due to the radioactive material can be determined. Therefore, medication treatment, surgical treatment, etc., according to the risk can be provided, enabling to inhibit the development of cancer.

### BRIEF DESCRIPTION OF THE DRAWINGS

FIG. 1 is a front cross-sectional view showing an embodiment of a diagnostic system for carcinogenicity of a first invention.

FIG. 2 is a diagram equivalent to a view on arrow II in FIG. 1 and showing a ring-shaped detector of a detecting apparatus in the system in FIG. 1.

FIG. 3 is a diagram corresponding to FIG. 2 and showing another example of the ring-shaped detector.

### DETAILED DESCRIPTION OF THE PREFERRED EMBODIMENTS

A diagnostic system for carcinogenicity of a first invention that diagnoses a risk of developing cancer due to a radioactive material deposited in a human body includes: a detecting unit that detects radiations emitted from the radioactive material; and a determining unit that obtains a dose of the radiations detected by the detecting unit and determines the risk based on the dose.

A diagnostic method for carcinogenicity of a second invention that diagnoses a risk of developing cancer due to a radioactive material deposited in a human body includes: a detection step of detecting radiations emitted from the radioactive material; and a determination step of obtaining a dose of the radiations detected in the detection step and determining the risk based on the dose.

The detecting unit is preferably a positron CT or a spectrometer. The positron CT and the spectrometer include radiation detectors.

The radioactive material is mainly radium deposited on a body which is formed by any one of ferritin and hemosiderin or both being deposited on an iron-containing substance. The iron-containing substance is mainly asbestos and the body to be formed is an asbestos body.

That is, when iron-containing amphibole asbestos, e.g., amosite or crocidolite, is taken into the human body, ferritin and hemosiderin which are present everywhere in the human body and which are known as iron-binding proteins are deposited on the asbestos, forming asbestos bodies. On the asbestos bodies, a radioactive material such as radium is deposited. In particular, since radium is deposited on the asbestos bodies at a relatively high density, the frequency of emission of radiation emitted from the radium increases. Accordingly, asbestos bodies having radium deposited thereon promote canceration of normal cells.

In addition, when an iron-containing substance other than asbestos, e.g., an iron-containing dust or gas, is taken into the human body, too, ferritin and hemosiderin are deposited on the iron that is contained in the dust or gas, forming bodies. On the bodies, a radioactive material such as radium is deposited. Therefore, such bodies cause normal cells to become cancerous, as with asbestos bodies. Note that examples of the iron-containing dust and gas include cigarette smoke and air in a mine or in a steelmaking plant, and thus, the possibility of inhalation is relatively high even in daily life.

In the present invention, the detecting unit detects radiations emitted from a radioactive material such as radium which is deposited on a body. That is, the detection step is performed by the detecting unit. Then, the determining unit obtains a dose of the detected radiations and determines, based on the dose, the risk of developing cancer due to the radioactive material. Namely, the determination step is performed by the determining unit. Such a determination is made by, for example, comparing the dose of the detected radiations with a dose threshold which is determined in advance from experience.

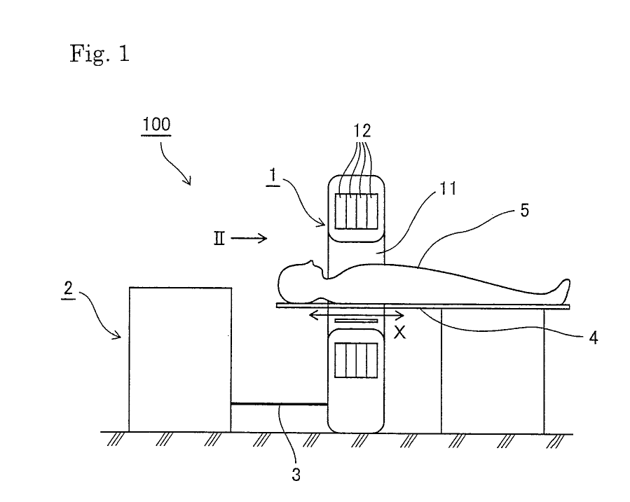

FIG. 1 is a front cross-sectional view showing an embodiment of the diagnostic system for carcinogenicity of the first invention. The diagnostic method for carcinogenicity of the second invention can be performed using this diagnostic system 100 for carcinogenicity. The diagnostic system 100 for carcinogenicity diagnoses the risk of developing cancer due to a radioactive material which is deposited in the human body. It is assumed that the radioactive material is radium and the radiation is γ rays. Specifically, the system 100 includes a detecting apparatus 1 that detects γ rays emitted from radium which is deposited in the human body; and a determining apparatus 2 that obtains a dose of the γ rays detected by the detecting apparatus 1 and determines the aforementioned risk based on the dose. The detecting apparatus 1 and the determining apparatus 2 are connected to each other by a wiring line 3. The detecting apparatus 1 is a cylindrical apparatus having a hollow 11. A bed 4 can move within the hollow 11 in an axial direction (X direction) with the bed carrying a test subject 5.

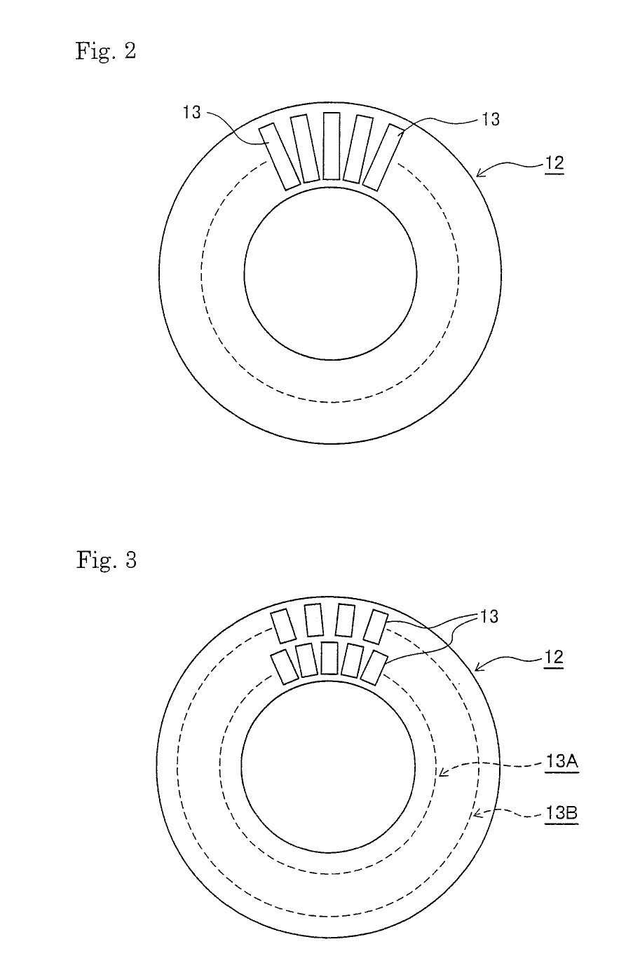

The detecting apparatus 1 is specifically a positron CT. As shown in FIG. 1, the positron CT is configured by numbers of ring-shaped detectors 12 being stacked together in the axial direction. Each ring-shaped detector 12 is formed such that, for example, as shown in FIG. 2 which is a diagram equivalent to a view on arrow II in FIG. 1, numbers of radiation detectors 13 are arranged in a ring configuration. The detecting apparatus 1 detects radiations by the respective ring-shaped detectors 12 and outputs detection signals to the determining apparatus 2.

The determining apparatus 2 includes a receiving unit that receives detection signals which are outputted from the detecting apparatus 1 through the wiring line 3; a counting unit that counts γ rays, based on the received detection signals; an estimating unit that estimates, based on a result of the count, a dose of γ rays emitted from radium which is deposited in the human body; a risk determining unit that determines the risk of developing cancer due to the radium, based on the estimated dose; and an identifying unit that obtains, based on the received detection signals, detected positions of γ rays which are detected substantially simultaneously by the detecting apparatus 1, identifies three or more flying directions of γ rays based on the detected positions, and identifies a radiation source based on the flying directions.

The risk determining unit shows the risk in, for example, three stages, “determination A”, “determination B”, and “determination C”. Specifically, when the dose of γ rays is less than or equal to a first threshold, the risk determining unit determines that there is almost no risk of developing cancer and thus provides “determination A”. When the dose of γ rays is greater than the first threshold and less than or equal to a second threshold, the risk determining unit determines that there is a small risk of developing cancer and thus provides “determination B”. When the dose of γ rays is greater than the second threshold, the risk determining unit determines that there is a high risk of developing cancer and thus provides “determination C”. Note that the first and second thresholds in this case are determined in advance from experience. Note also that “determination B” indicates “follow-up required”, i.e., indicates that the test subject is recommended to undergo semi-annual or annual reexamination, and “determination C” indicates that the test subject is recommended to get medication treatment or surgical treatment.

The identifying unit is activated when the risk determining unit provides “determination C”. The identifying unit obtains detected positions of γ rays which are detected substantially simultaneously by the ring-shaped detectors 12 adjacent to each other in the detecting apparatus 1, identifies three or more flying directions of γ rays based on the detected positions, and identifies a radiation source based on the flying directions. Identification of a radiation source based on flying directions is specifically performed as follows. Specifically, imaginary straight lines are drawn along the respective identified flying directions of γ rays, a position of an intersection of three or more imaginary straight lines is obtained, and the position of the intersection is determined as the position of a radiation source. Note that in practice since three or more imaginary straight lines do not intersect at one point, a central position in a region with high intersection density is determined as the position of a radiation source.

As described above, the system 100 shown in FIG. 1 can detect γ rays emitted from radium which is deposited in the human body, obtain a dose of the γ rays, and determine the risk of developing cancer due to the radium, based on the dose. Thus, by appropriately providing treatment according to the risk, the development of cancer can be prevented. In particular, the development of cancer caused by asbestos can be prevented.

Note that a positron CT which is used as the detecting apparatus 1 may be configured by using ring-shaped detectors 12 shown in FIG. 3. In the ring-shaped detector 12 in FIG. 3, radiation detectors 13 are arranged in a double-ring configuration including an inner row 13A and an outer row 13B. When this detecting apparatus 1 is used, the position of a radiation source can be identified more accurately by the determining apparatus 2.

Although, in the above-described system 100, a positron CT is used as the detecting apparatus 1, a spectrometer, a Geiger counter, etc., may be used instead.

A diagnostic system for carcinogenicity of the present invention can diagnose the risk of developing cancer and thus has great value in industrial use.

What is claimed is:

1. A diagnostic system for carcinogenicity that diagnoses a risk of developing cancer due to a radioactive material deposited in a human body, the system comprising:
a detecting unit that detects radiations emitted from the radioactive material; and
a determining unit that obtains a dose of the radiations detected by the detecting unit and determines the risk based on the dose.

2. The diagnostic system for carcinogenicity according to claim 1, wherein the detecting unit is a positron CT or a spectrometer.

3. The diagnostic system for carcinogenicity according to claim 2, wherein the determining unit further identifies three or more flying directions of radiations, based on detected positions of radiations which are detected substantially simultaneously by the detecting unit, and identifies a radiation source based on the flying directions.

4. The diagnostic system for carcinogenicity according to claim 2, wherein the determining unit includes:
a receiving unit that receives detection signals from the detecting unit;
a counting unit that counts γ rays, based on the received detection signals;
an estimating unit that estimates, based on a result of the count, a dose of γ rays emitted from radium which is deposited in the human body;
a risk determining unit that determines a risk of developing cancer due to the radium, based on the estimated dose; and
an identifying unit that obtains, based on the received detection signals, detected positions of γ rays which are detected substantially simultaneously by the detecting unit, identifies three or more flying directions of γ rays based on the detected positions, and identifies a radiation source based on the flying directions.

5. The diagnostic system for carcinogenicity according to claim 1, wherein the radioactive material is radium deposited on a body which is formed by any one of ferritin and hemosiderin or both being deposited on an iron-containing substance.

6. The diagnostic system for carcinogenicity according to claim 5, wherein the iron-containing substance is asbestos.

7. The diagnostic system for carcinogenicity according to claim 5, wherein the iron-containing substance is dust or a gas.

8. A diagnostic method for carcinogenicity that diagnoses a risk of developing cancer due to a radioactive material deposited in a human body, the method comprising:
a detection step of detecting radiations emitted from the radioactive material; and
a determination step of obtaining a dose of the radiations detected in the detection step and determining the risk based on the dose.

9. The diagnostic method for carcinogenicity according to claim 8, wherein in the determination step, three or more flying directions of radiations are further identified based on detected positions of radiations which are detected substantially simultaneously in the detection step, and a radiation source is identified based on the flying directions.

10. The diagnostic method for carcinogenicity according to claim 8, wherein the determination step includes:
a reception step of receiving detection signals obtained in the detection step;
a counting step of counting γ rays, based on the received detection signals;
an estimation step of estimating, based on a result of the count, a dose of γ rays emitted from radium which is deposited in the human body; p1 a risk determination step of determining a risk of developing cancer due to the radium, based on the estimated dose; and
an identification step of obtaining, based on the received detection signals, detected positions of γ rays which are detected substantially simultaneously in the detection step, identifying three or more flying directions of γ rays based on the detected positions, and identifying a radiation source based on the flying directions.
